(19) United States
(12) Patent Application Publication
(10) Pub. No.: US 2007/0088201 A1
(43) Pub. Date: Apr. 19, 2007

(54) Endoscope And Method For Its Manufacturing

(76) Inventor: Peter Eisenkolb, Tuttlingen (DE)

Correspondence Address: Wesley W. Whitmyer, Jr.;ST.ONGE STEWARD JOHNSTON & REENS LLC, 986 Bedford Street, Stamford, CT, 06905-5619, US

(21) Appl. No.: 11/550,719

(22) Filed: Oct. 18, 2006

(30) Foreign Application Priority Data
Oct. 18, 2005 (DE) 10 2005 051 208.9

(51) Int. Cl. A61B 1/06; A61B 1/00

(52) U.S. Cl. 600/182; 600/920; 600/130; 600/138

(57) ABSTRACT

An endoscope has a shaft and an endoscope housing a proximal end of said shaft. A light guide connector projects laterally from the endoscope head. A light guide composed of a bundle of light guiding fibers is guided in said shaft from a distal end to a proximal end thereof followed by a curvature to the laterally extending light guide connector. The bundle of light guiding fibers is sheathed in the area of said curvature by application of a flexible adhesive.

### BACKGROUND OF THE INVENTION

This invention relates to an endoscope having a shaft and an endoscope head housing a proximal end of said shaft. A light guide connector projects laterally from said endoscope head. A light guide composed of a bundle of light guiding fibers is guided in said shaft from a distal end to a proximal end thereof. Within the endoscope head the light guide is bent laterally via a curvature to the laterally extending light guide connector.

The invention also relates to a method for manufacturing such an endoscope.

Endoscopes of this type are generally known.

A general use of endoscopes is during so-called minimal-invasive surgeries

These are for example arthroscopy, gastro-intestinal examinations and chest cavity examination, for example in the case of ruptures and during joint and spinal column surgeries.

Endoscopes of this kind usually have a thin elongated endoscope shaft.

The endoscope shaft is commonly composed of two tubes. An outer tube has inserted coaxially an inner tube with a smaller diameter.

At its proximal end, the endoscope shaft is mounted in an endoscope head.

A so-called optic composed of a rod-lens system or an electronic image recording system is housed within the inner tube.

A group of optical light guides is provided loosely in the endoscope shaft between the outer side of the inner tube and the inner side of the outer tube. The optical light guides are usually composed of a bundle of individual fibers. This fibers are usually made of glass, however it can also be produced from multi-component glass or plastic.

The light guide serves for guiding light from the lateral light guide connector to the distal end of the shaft for illuminating the operation site.

Within the endoscope bead, the light guides passing the proximal end of the shaft are guided via curvature to the lateral extending light connector.

This area has been found to be problematic during practical use.

Fine fracture cracks can be formed either at the distal end of the shaft or the proximal end of the light guide connector. Moisture can enter into the endoscope head through these cracks. Furthermore, chemically aggressive cleaning substances, for example peroxide containing substances, which are used for sterilization purposes can enter the endoscope head in this way.

In consequence, the light guides can be attacked and damaged during autoclaving cycles, in particular in the curved area between proximal end of shaft and light guide connector. This increases the risk of fiber fracture in particular in the curved area within the endoscope head, resulting in the endoscope having a reduced lifetime.

It is therefore an object of the invention to provide an endoscope having a long lifetime.

It is further an object of the invention to provide a method for manufacturing an endoscope having a long lifetime.

### SUMMARY OF THE INVENTION

According to the invention this object is achieved by an endoscope having a shaft, an endoscope head housing a proximal end of said shaft, a light guide connector projecting laterally from said endoscope head, a light guide composed of a bundle of light guiding fibers, said light guide being provided in said shaft from a distal end thereof to a proximal end thereof, followed by a curvature to the laterally extending light guide connector, wherein said bundle of light guiding fibers is sheathed in the area of said curvature by application of a flexible adhesive.

In the method according to the invention the bundle of light guide fibers is, in the area of the curvature sheathed by application of a flexible adhesive to said bundle. When curing the adhesive the bundle is enveloped by that cured but still flexible adhesive in the entire area of said curvature.

The light guide is now protected by the outer sheath of cured and flexible adhesive resting closely on it. At the same time, the liquid adhesive which has penetrated at least in the outer circumferential area of the bundles links the individual fibers in that circumferential area in kind of a composite material.

The resulting curved section of the light guide is still flexible which means, that mechanical shocks or thermal stresses loaded on that curvature can be taken without the risk of cracking or damaging the individual fibers. This results in a remarkable longer lifetime of the endoscope.

In a refinement, the flexible adhesive is impermeable to moisture.

This has the advantage, that the protective sheath provides the optical fibers against any kind of moisture which may possibly enter the interior of the endoscope head.

This remarkably prolongs the lifetime.

In a further refinement the flexible adhesive is resistant to chemicals.

This has the advantage that the flexible adhesive keeps any chemical aggressive substance which can enter the endoscope head away from the optical light guide. The adhesive acts as a barrier and protects the individual fibers from environmental influences.

In a further refinement of the invention, beyond the two opposite ends of the curvature, i.e. the proximal end of the light guide connector and the inlet into the proximal end of the endoscope shaft, the light guide is provided with the flexible adhesive, too.

This measure has the advantage, that the opposite ends of the curvature are sealingly fitted via the adhesive to the proximal end of the light connector on the one side and to the inlet cross section of the proximal end of the shaft on the other side. This prevents moisture entering from the outer side via the light connector or, if otherwise entered into the endoscope head, a further penetration into the shaft is avoided.

If moisture penetrates the shaft from its distal end, a passing into the head is avoided, too.

In a further refinement of the invention the individual fibers of the light guide are likewise sheathed with the flexible adhesive.

This measure has the advantage, that not only the outer peripheral fibers are protected, but also all of the individual fibers of the bundle. The individual fibers are now embedded in the cured but still flexible adhesive and are therefore protected against any outer actions. These actions may be mechanical loads or attacks by moisture or chemical aggressive liquids.

According to refinements of the method of the invention, the liquid adhesive before curing it can be applied in such a manner, that it either covers only the outer circumferential area of the bundle in the curvature or, all of the individual fibers are wetted with the still liquid adhesive.

If the adhesive also serves as a seal of the opposite ends of the curvature, it is applied in such that the adhesive penetrates at least the proximal end area of the shaft between inner and outer tube and the proximal end of the light guide connector.

After curing the adhesive a kind of composite body is created composed of the individual fibers embedded in the cured but still flexible adhesive which can also seal the opposite ends of the curvature.

It is self-evident that the features mentioned above and those still to be explained in the following text can be used not only in the respectively stated combination but also in other combinations or on their own without departing from the scope of the present invention.

### BRIEF DESCRIPTION OF THE DRAWINGS

The invention will be described and explained in more detail in the following text with reference to an exemplary embodiment in consumption with the attached drawings in which:

FIG. 1 shows a perspective view of an endoscope according to the invention;

FIG. 2 shows partly a longitudinal section through the endoscope of FIG. 1;

FIG. 3 schematically shows a section along the line III-III in FIG. 2; and

FIG. 4 shows a comparable section to that in FIG. 3 of an further embodiment having all of the individual fibers of the bundle impregnated and embedded with adhesive.

### DETAILED DESCRIPTION OF PREFERRED EMBODIMENTS

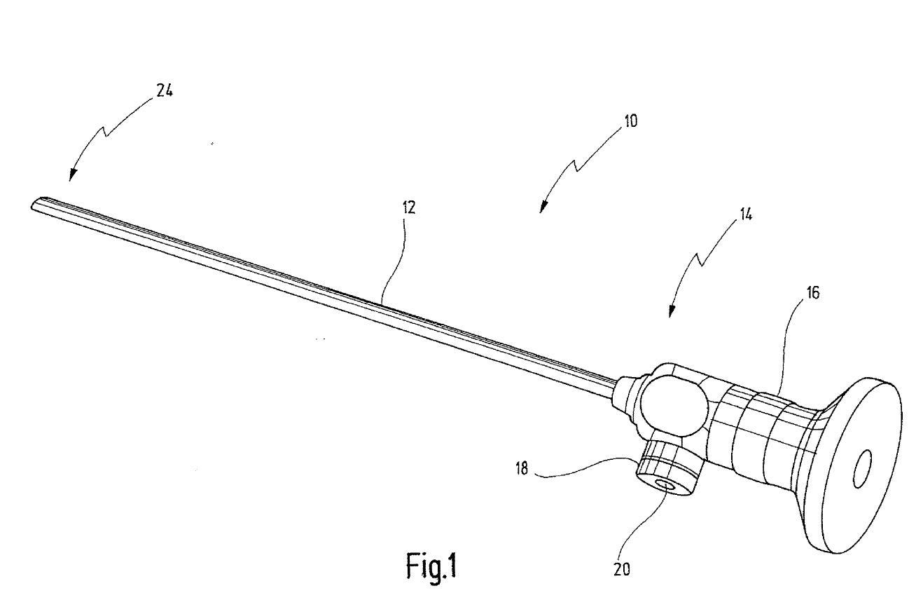

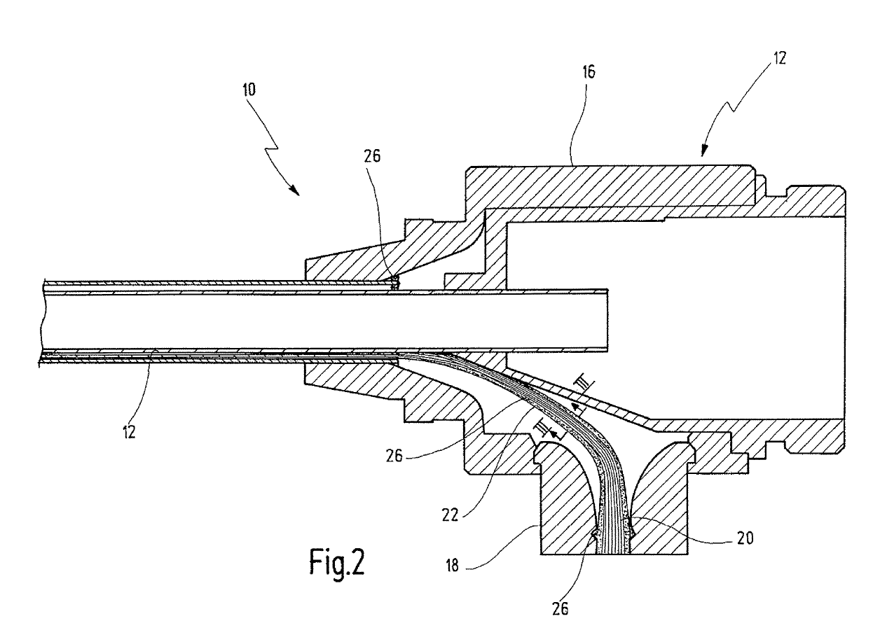

FIG. 1 shows an endoscope which is annotated in its totality with reference number 10. The endoscope 10 has an endoscope shaft 12. The endoscope shaft 12 is held at its proximal end 14 in an endoscope head. The endoscope head 16 has a laterally projecting light guide connector 18. A proximal end of the endoscope head 16 is provided with an ocular which is not shown in the section of FIG. 2.

As shown in FIG. 2 an optical light guide 20 is housed in the proximal end of the light guide connector 18. The optical light guide 20 is in form of a bundle of individual glass fibers.

As also can be seen from FIG. 2, the optical light guide 20 is guided from the proximal end of the light guide connector 18 via a curvature 22 to the proximal end of the endoscope shaft 12.

The optical light guide 20 extends up to the distal end 24 of the endoscope shaft 12. The shaft 12 is composed of an outer tube and a coaxially inserted inner tube. The optical light guide 20 is housed within the space between the outer side of the inner tube and the inner side of the outer tube.

The optical light guide 20 is sheathed with a flexible adhesive 26 in the curved area between the laterally projecting light guide connector 18 and the proximal end of the endoscope shaft 12.

The flexible adhesive 26 is impermeable to moisture and is resistant to chemicals.

Further, both a mouth opening of the waveguide connector 18 and an inlet cross section at the proximal end of the endoscope shaft 12 are provided with the adhesive 26 and are thus sealed.

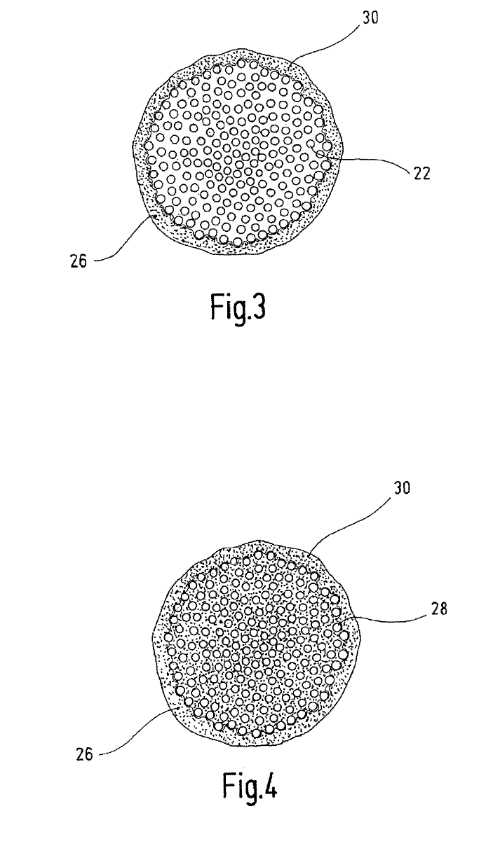

FIG. 3 shows, that the optical light guide 20 is composed of individual fibers 22. The flexible adhesive 26 is applied in this embodiment to the bundle of individual fibers 22 as an outer protective sheath 30. The protective sheath 30 is closely on it and is penetrated to a certain extend into the intermediate spaces between the outer fibers 22. The fibers 22 within the interior of the bundle can still move along one another.

FIG. 4 shows a further embodiment. In this embodiment, the flexible adhesive 26 is applied in that it penetrates the intermediate spaces between the individual fibers 28 also within the inner core area of the bundle. This can be achieved by way of using a flexible adhesive with low viscosity. Such a low viscosity liquid adhesive can penetrate up to the inner core area of the bundle of fibers resulting in an embedding of all of the individual fibers of the bundle with adhesive. After curing all of the fibers 28 are embedded within cured adhesive 26. Nevertheless, an outer closed sheath 30 of cured flexible adhesive is present, which covers the bundle on its outer side.

During assembly of the endoscope 10, the optical wave guide, i.e. the bundle of glass fibers is posed in the interior in that it is inserted into the space within the shaft 12 and fed from the proximal end via a curvature to the laterally extending light guide connector 18. The endoscope head is designed as multiple part head, allowing access to the curvature 22 of the bundle of fibers. Via this access an adhesive 26 can be applied for example via a brushing apparatus or a kind of syringe. The amount of adhesive 26 applied to the bundle depends on how the resulting sheath should be.

If one wants to result the embodiment of FIG. 3, one uses a more viscous adhesive which covers the entire outer surface of the bundle within the curved area without penetrating remarkably into it.

If one wants to achieve the embodiment shown within FIG. 4, one uses a more viscous adhesive which can penetrate up to the inner core of the bundle of fibers. In that case, it may be possible to serially apply the adhesive up to all of the adhesive has penetrated the bundle and followed by a final outer appliance to ensure the outer sheath 30. After curing the adhesive a still flexible but tightly sheaths the bundle of glass fibers in the area of its curvature 22.

What is claimed is:

1. An endoscope having
a shaft,
an endoscope head housing a proximal end of said shaft,
a light guide connector projecting laterally from said endoscope head,
a light guide composed of a bundle of light guiding fibers, said light guide being guided in said shaft from a distal end thereof to a proximal end thereof followed by a curvature to the laterally extending light guide connector, wherein
said bundle of light guiding fibers is sheathed in an area of said curvature by application of a flexible adhesive.

2. The endoscope of claim 1, wherein said flexible adhesive is impermeable to moisture.

3. The endoscope of claim 1, wherein said flexible adhesive is resistant to chemicals.

4. The endoscope of claim 1, wherein beyond opposite ends of said curvature, i.e. a proximal end of said light guide connector and an inlet cross section of a proximal end of said shaft, said light guide is provided with said flexible adhesive, too.

5. The endoscope of claim 1, wherein individual fibers of said optical light guide are sheathed with the flexible adhesive.

6. Method for preparing an endoscope having
a shaft,
an endoscope head housing a proximal end of said shaft,
a light guide connector projecting laterally from said endoscope head,
a light guide composed of a bundle of light guiding fibers, said light guide being guided in said shaft from a distal end thereof to a proximal end thereof followed by a curvature to the laterally extending light guide connector, wherein
said bundle of light guiding fibers is sheathed in an area of said curvature by application of a flexible adhesive and followed by a curing of said adhesive.

7. The method of claim 6, wherein said adhesive is also applied into an inlet cross section of said bundle of fibers of the proximal end of said shaft.

8. The method of claim 6, wherein said adhesive is applied in such that individual light guide fibers are likewise sheathed with that flexible adhesive.
